(19) United States
(12) Patent Application Publication
(10) Pub. No.: US 2003/0138859 A1
(43) Pub. Date: Jul. 24, 2003

(54) Assay kits and methods for immune complex-mediate activation involving shed antigens

(75) Inventors: Emilio Barbera-Guillem, Powell, OH (US); M. Bud Nelson, Worthington, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(73) Assignee: BioCrystal, Ltd., Westerville, OH

(21) Appl. No.: 10/289,732

(22) Filed: Nov. 7, 2002

Related U.S. Application Data
(63) Continuation of application No. 09/541,747, filed on Apr. 3, 2000, now abandoned.
(60) Provisional application No. 60/127,689, filed on Apr. 5, 1999.

(51) Int. Cl. G01N 33/53; G01N 33/567

(52) U.S. Cl. 435/7.21

(57) ABSTRACT

Methods and assay kits are provided for assaying for a measurable biological response inducible by activation of FcγRI-expressing cells by immune complexes comprising shed antigen and anti-shed antigen antibody of an IgG subtype capable of binding FcγRI. An assay kit comprises shed antigen, anti-shed antigen antibody of an IgG subtype capable of binding FcγRI, and FcγRI-expressing cells.

This application is a nonprovisional based on earlier, co-pending provisional application Serial No. 60/127,689, the disclosure of which is herein incorporated by reference.

### FIELD OF THE INVENTION

The present invention is related to novel in vitro assays which may be used qualitatively or quantitatively to assess or screen for inducers, as well as to screen for inhibitors, of immune complex-mediated disease processes. More particularly, the present invention is related to assays which are based on shed antigen-containing immune complexes that bind and cross-link Fc gamma receptor (FcγRI) on FcγRI-expressing cells in activating the cells to produce a measurable biological response. The assays can be used to screen for and identify such immune complexes (“inducers”), as well as to screen for inhibitors of the activation of the FcγRI-expressing cells by such immune complexes.

### BACKGROUND OF THE INVENTION

Fc gamma receptors are cell surface glycoproteins divided into three main types based on criteria including molecular mass, specificity and affinity for the different subtypes of IgG, and reactivity with monoclonal antibodies. It is known that the FcγR type will affect the IgG subtypes for which it has binding specificity and avidity, which location (s) of the IgG Fc portion is bound, and what subsequent immune killing or regulatory function or activation process is mediated by such binding to the FcγR type. The three types of Fcγ receptors include FcγRI (CD64), FcγRII (IIA, IIB) (CD32), and FcγRIII (IIIA, IIIB) (CD16). Human FcγRII is the most widely expressed FcγR, including expression by neutrophils, B cells, T cells, platelets, macrophages, monocytes, epithelial cells, endothelial cells, and epidermal Langerhans cells. FcγRIII is expressed by gamma-delta T cells, epidermal Langerhans cells, natural killer (NK) cells, neutrophils, differentiated monocytes, and macrophages. In contrast, FcγRI is expressed only on a limited number of cells, and typically only upon cytokine-mediated induction of expression. FcγRI expression is “virtually undetectable on mature polymorphonuclear neutrophils (PMNs) in healthy individuals” (Gericke et al., 1995, J. Leukoc. Biol. 57:455-61); and has not been characterized on vascular endothelial cells. The FcγR expressed by tumor cells has been identified as FcγRIIB, based on reactivity with monoclonal antibody 2.4G2 having binding specificity for FcγRIIB (see, e.g., Ran et al., 1984, J. Natl. Cancer Inst. 73:437-45; 1988, Mol. Immunol. 25:1159-67).

The present invention relates to the discovery that shed antigen-containing immune complexes activate FcγRI-expressing cells in inducing a biological response that is related (the response indirectly or directly contributes) to disease progression in vivo; and that the biological response can be reproduced, identified, and assayed in vitro. Thus, there is a need for in vitro assays and methods useful for screening substances (e.g., new types of shed antigen-containing immune complexes) which activate FcγRI-expressing cells; or for screening substances (e.g., a pharmaceutically effective amount of a therapeutic inhibitor or drug) which inhibit activation of FcγRI-expressing cells by shed antigen-containing immune complexes.

### SUMMARY OF THE INVENTION

Accordingly, it is a primary object of the present invention to provide an in vitro method for performing activation of FcγRI-expressing cells by immune complexes comprising shed antigen and anti-shed antigen antibody of IgG subtype capable of binding FcγRI.

It is another object of the present invention to provide an in vitro method of quantitatively assaying inhibitors of the activation of FcγRI-expressing cells by these immune complexes.

It is another object of the present invention to provide an in vitro method for performing activation of FcγRI-expressing cells by these immune complexes, wherein the FcγRI-expressing cells are tumor cells, and wherein the biological response resulting from activation comprises one or more of induction of tyrosine kinase (e.g., amount and/or activity), induction of tumor cell proliferation, and induction of shed antigen production, by the tumor cells.

It is another object of the present invention to provide an in vitro method of assessing (qualitatively or quantitatively) the ability of, or screening (qualitatitively, or quantitatively) for, inhibitors of the activation of FcγRI-expressing cells by these immune complexes, wherein the FcγRI-expressing cells are tumor cells, wherein assayed for is inhibition of the biological response which would result from activation, and wherein the biological response comprises one or more of induction of tyrosine kinase (e.g., amount and/or activity), induction of tumor cell proliferation, and induction of shed antigen production, by the tumor cells.

It is another object of the present invention to provide an in vitro method for performing activation of FcγRI-expressing cells by these immune complexes, wherein the FcγRI-expressing cells are immune effector cells, and wherein the biological response resulting from activation comprises one or more of induction of tyrosine kinase, of cytokine production, of cell degranulation, and of degradative enzyme release (e.g., amount and/or activity), by the immune effector cells.

It is another object of the present invention to provide an in vitro method of assessing the ability of, or screening for, inhibitors of the activation of FcγRI-expressing cells by these immune complexes, wherein the FcγRI-expressing cells are immune effector cells, and wherein assayed for is inhibition of the biological response resulting from activation, and wherein the biological response comprises one or more of induction of induction of tyrosine kinase, of cytokine production, of cell degranulation, and of degradative enzyme release (e.g., amount and/or activity), by the immune effector cells.

It is a further object of the present invention to provide an in vitro method for performing activation of FcγRI-expressing cells by these immune complexes, wherein the FcγRI-expressing cells are endothelial cells, and where-in the biological response resulting from activation com-prises one or more processes related to angiogenesis (e.g., as detectable by one or more of induction of tyrosine kinase, of cytokines (and in a preferred embodiment, vascular endothelial growth factor) production, and of cell proliferation, by the endothelial cells.

It is another object of the present invention to provide an in vitro method of assessing the ability of, or screening for, inhibitors of the activation of FcγRI-expressing cells by these immune complexes, wherein the FcγRI-expressing cells are endothelial cells, wherein assayed for is inhibition of the biological response resulting from activation, and wherein the biological response by the endothelial cells comprises one or more processes related to angiogenesis.

The foregoing objects are achieved by identifying

(a) a novel mechanism by which a shed antigen induces a humoral immune response which results in the production of anti-shed antigen antibody including of the IgG subtype capable of binding FcγRI;

(b) a novel mechanism by which immune complexes comprising shed antigen and anti-shed antigen antibody of IgG subtype can bind and cross-link FcγRI on FcγRI-expressing cells in vivo, wherein as a result of the cross-linking the cells are activated to produce a biological response that may be related (e.g., indirectly or directly the response may contribute) to disease progression in vivo; and

(c) that the biological response can be sufficiently reproduced, identified, and assayed in vitro so as to provide assays which may be used to screen for and identify such immune complexes that can activate, as well as to screen for inhibitors of such activation of, FcγRI-expressing cells. More particularly, immune complexes comprising shed antigen and anti-shed antigen antibody of IgG subtype capable of binding FcγRI (and preferably of the IgG1 subtype) can bind FcγRI on FcγRI-expressing cells in a process of promoting one or more of cell proliferation, tissue degradation, tissue invasion, and subsequent immune-complex mediated disease progression. As will be described in more detail herein, the shed antigen is a molecule which is released or secreted from cells and can induce a humoral immune response including production of IgG1 antibodies against shed antigen; contains an immunostimulatory number of antigenic determinants which can be spatially presented for binding by a plurality of antibody molecules thereby resulting in immune complexes having a threshold level for spacing, and an optimal number of, antibody molecules for FcγRI binding and cross-linking on FcγRI-expressing cells. The FcγRI-expressing cells comprise one or more of FcγRI-expressing immune effector cells, FcγRI-expressing tumor cells, and FcγRI-expressing endothelial cells (as opposed to lacking detectable FcγRI-expression). The cross-linking of FcγRI on these FcγRI-expressing cells may result in a signal that activates the cells to induce production of substances and/or processes which mediate disease progression (a “biological response”). In an illustrative, non-limiting example, cross-linking of FcγRI on these FcγRI-expressing cells results in the activation of protein-tyrosine kinases which in turn may lead to induction of cytokine production (e.g., by cross-linked immune effector cells and by cross-linked endothelial cells), to induction of shed antigen production (e.g., by cross-linked tumor cells), and/or induction of cell proliferation (e.g., by cross-linked tumor cells and cross-linked endothelial cells).

The above and other objects, features, and advantages of the present invention will be apparent in the following Detailed Description of the Invention when read in conjunction with the accompanying drawings.

### BRIEF DESCRIPTION OF THE DRAWINGS

FIG. 1 is a bar graph illustrating the liver metastasis scores in mice treated with either saline, irrelevant goat IgG, or goat anti-mouse IgG.

FIG. 2 is a bar graph showing the number of T-47D tumor cells plotted in relation to various concentrations of anti-sTn antibody.

FIG. 3 is a bar graph showing relative number of B16F10 melanoma cells plotted in relation to various concentrations of anti-sTn antibody either in the presence of added mucin or the absence of added mucin.

FIG. 4 is a graph showing the ratio of the amount of mucin produced to cell proliferation, plotted in relation to the concentration of anti-sTn IgG1 mAb.

FIG. 5 is a bar graph illustrating invasion of shed tumor antigen-secreting tumor cells through matrix when incubated with various cellular components, antibodies, or antibody fragments.

### DETAILED DESCRIPTION OF THE INVENTION

Definitions

The term “activation” is used herein, for purposes of the specification and claims, and in reference to immune complexes comprising shed antigen and anti-shed antigen antibody of IgG subtype capable of binding FcγRI (preferably of IgG1 subtype) binding to and cross-linking FcγRI on FcγRI-expressing cells, to mean induction of a signal transduction pathway that leads to the induction of a measurable biological response by the cross-linked cells, as will be more apparent from the following definitions and descriptions.

The term “induction” is used herein, with reference to a biological response, and for purposes of the specification and claims, to mean the measured biological response produced as a result of activation is comparatively greater (e.g., an increase in amount, or activity, or number, depending on the biological response measured) than the comparative biological response produced (if any) from comparative FcγRI-expressing cells not activated by the immune complexes. Inducers comprise shed antigens, which when complexed with the appropriate anti-shed antigen antibody of IgG subtype capable of binding FcγRI in forming immunostimulatory immune complexes, can result in induction.

The term “inhibitor of activation” is used herein, for purposes of the specification and claims, to mean a substance which inhibits one or more steps in the activation process, wherein the steps may comprise (a) binding of immune complexes (comprising shed antigen and anti-shed antigen antibody of IgG subtype capable of binding FcγRI (in a preferred embodiment, IgG1) to FcγRI on FcγRI-expressing cells; (b) cross-linking of the FcγRI on FcγRI-expressing cells by such immune complexes (the resultant activated cells, having been activated by the cross-linking of FcγRI thereon, are referred to herein, for purposes of brevity only, as “cross-linked cells”); (c) activation of a signal transduction pathway in the cross-linked cells; (d) gene activation (e.g., induction of expression) as a result of the activation of a signal transduction pathway in the cross-linked cells; (e) depending on the cell type which is cross-linked, induction of cytokine production, as will be more apparent from the following descriptions; (f) depending on the cell type which is cross-linked, induction of shed antigen production (e.g., wherein the type of cross-linked cells comprises tumor cells); and (g) depending on the cell type which is cross-linked, induction of cell proliferation (e.g., wherein the type of cross-linked cells comprises a type selected from the group consisting of tumor cells, endothelial cells, and a combination thereof; wherein a combination thereof refers to a mixed population of cell types; e.g., a combination of tumor cells and endothelial cells). An inhibitor may be inhibiting activation at a specific step. As an illustrative example, to “inhibit” the immune complexes from binding to FcγRI on FcγRI-expressing cells may mean blocking the immune complexes (preferably containing IgG1) from subsequently binding to FcγRI on the FcγRI-expressing cells so as to minimize or prevent this step in the activation process (e.g., by a peptide or antibody or antibody fragment such as an Fv or Fab, which specifically binds to the FcγRI in competing with the immune complexes for binding to FcγRI) As an illustrative example, to “inhibit” the immune complexes from cross-linking of the FcγRI on FcγRI-expressing cells may mean blocking all or a sufficient number of the FcγRI on FcγRI-expressing cells so as to minimize or prevent cross-linking of the FcγRI by the immune complexes (e.g., by a peptide or antibody or antibody fragment such as an Fv or Fab, which binds to the FcγRI in competing with the immune complexes for cross-linking the FcγRI). As an illustrative example, to “inhibit” the activation of a signal transduction pathway in the cross-linked cells may mean one or more of preventing or blocking the signal transduction pathway from being activated by the FcγRI cross-linking process; or inactivating or down regulating the signal transduction pathway, after it has been activated by the FcγRI cross-linking process, so as to minimize or prevent it from inducing a biological response such as inducing gene activation or inducing cytokine production, or inducing cell proliferation (e.g., if the signal transduction pathway comprises tyrosine kinase, an inhibitor may comprise a tyrosine kinase inhibitor as known in the art).

The term “inhibit” is used herein, with reference to induction of a measurable biological response by cross-linked cells, and for the purposes of the specification and claims, to mean that the measured biological response produced as a result of activation is comparatively less (e.g., a decrease in amount, activity, or number, depending on the biological response measured) in the presence of an inhibitor than the comparative biological response produced from comparative cross-linked cells in the absence of the inhibitor. In a preferred embodiment, the inhibition may be manifest as a level of a biological response which is equal to or greater than 2 standard deviations less than the comparative biological response produced from comparative cross-linked cells in the absence of the inhibitor.

The term “biological response” is used herein, for purposes of the specification and claims, to mean one or more of (a) a measurable activation of a signal transduction pathway in cross-linked cells; (b) measurable gene activation as a result of the activation of a signal transduction pathway in cross-linked cells; (c) depending on the cell type which is cross-linked, measurable induction of cytokine production; (d) depending on the cell type which is cross-linked, measurable induction of shed antigen production; and (e) depending on the cell type which is cross-linked, measurable induction of cell proliferation. A biological response may be assayed using any one of several methods known in the art for assaying the presence of that particular biological response, as will be more apparent from the following descriptions. A preferred biological response may be assayed to the exclusion of a biological response other than the preferred biological response. Likewise, a preferred biological response by a preferred cell type of FcγRI-expressing cells may be assayed to the exclusion of the biological response in a cell type other than the preferred cell type of FcγRI-expressing cells. For example, one preferred embodiment of the present invention is an assay which measures the induction of shed antigen production by FcγRI-expressing tumor cells (or, when a substance is added to test its ability to act as an inhibitor, assayed for is the inhibition of the induction of shed antigen production by FcγRI-expressing tumor cells).

The term “immune effector cells” is used herein, for purposes of the specification and claims, to mean a cell type selected from the group consisting of neutrophils, macrophages, astrocytes, microglia (astrocytes and microglia being intrinsic central nervous system immune effector cells), a cell line thereof, and a combination thereof (e.g., two or more cell types such as a combination of neutrophils and macrophages in the assay). In a preferred embodiment, the immune effector cells are of mammalian origin, and in a more preferred embodiment, are of human origin. A preferred cell type of immune effector cells may be assayed for a biological response to the exclusion of immune effector cells other than the preferred cell type of immune effector cells. In one preferred embodiment, the immune effector cells are selected from the group consisting of a cell line, cells isolated from an individual having a disease which has activated the individual's immune system, and a combination thereof. If desired, the FcγRI expression on immune effector cells may be upregulated before being assayed by pretreatment of the immune effector cells with pro-inflammatory cytokines in vitro such as tumor necrosis factor-alpha (TNF-α), interleukin-1 beta (IL-1p) of interferon-gamma (using methods well known in the art for pretreating cells with proinflammatory cytokines (e.g., incubation of the cells with 100 U/ml IFN-γ, or with 10 ng/ml TNF-α, or with 20 ng/ml IL-1β, for 24 hours in a physiological buffer). However, in a preferred embodiment, the immune effector cells are not pretreated in vitro with one or more proinflammatory cytokines.

The term “tumor cells” is used herein, for purposes of the specification and claims, to mean tumor cells of ductal epithelial cell origin, including, but not limited to, tumor cells originating in the liver, lung, brain, lymph node, breast, colon, pancreas, stomach, rectum, prostate, or reproductive tract (cervix, ovaries, endometrium etc.); and which produces shed antigen (e.g., serous, or endometroid, or mucinous tumors) which may also be referred to herein as “shed tumor antigen”; and may also include other FcγRI-expressing tumor cells such as melanoma cells or lymphoma cells. A preferred type (e.g., organ origin) tumor cells may be assayed for a biological response to the exclusion of tumor cells of a type other than the preferred tumor cells.

The term “FcγRI-expressing cells” is used herein, for purposes of the specification and claims, to mean a cell type comprising one (a single) or more (e.g., more than one cell type in a mixed population of cells) of tumor cells, immune effector cells, endothelial cells, or a cell line derived therefrom; and wherein the cell type expresses detectable amounts of one or more of the receptors in the FcγRI family (e.g., FcγRIA, FcγRIB, FcγRIC; Ernst et al., 1992, J. Biol. Chem. 267: 15692-15700; Porges et al., 1992, J. Clin. Invest. 90:2102-2109; or other isoform) on the cell surface which can function to bind immune complexes, as will be more apparent from the following descriptions.

The term “immune complex-mediated disease progression” is used herein, for purposes of the specification and claims, to mean a humoral immune response against a shed antigen that results in immune complexes formed between antibody (particularly of the IgG1 subtype) to shed antigen, and shed antigen. Such immune complexes, when present in vivo, may then promote disease progression by one or more mechanisms including: binding and cross-linking FcγRI on FcγRI-expressing cells resulting in activation and induction of, and release of, inflammatory mediators (e.g., one or more of cytokines, and shed antigen) which promote angiogenesis, and tissue invasion; and binding and cross-linking FcγRI on FcγRI-expressing cells resulting in activation of the cross-linked cells and induction of a biological response which contributes to disease progression.

The term “shed antigen” is used herein, for purposes of the specification and claims, to mean a glycomolecule (e.g., glycoprotein or glyclipid) which:

(a) by itself, or in an aggregated or oligomeric (two or more monomers which are together) form, has a molecular size equal to or greater than about 100 kilodaltons;

(b) is released from cells producing it, thereby becoming soluble and allowing for movement into tissues;

(c) comprises a molecule having repeated (more than one per molecule) carbohydrate chains which present a terminal, repeated carbohydrate epitope, wherein terminal carbohydrate epitope comprises a terminal 2-6 linked sialic acid such as sialyl Tn (sTn) antigen (substantially comprising the 2-6 linked NeuAc of NeuAcα2→6GalNAc α1→O-Ser- or Thr in mucins (e.g., Muc-1) shed by adenocarcinomas, or in human meconium glycoproteins, or in submaxillary mucins (e.g., of bovine, ovine or porcine origin)), or a sialic acid-containing epitope other than sTn antigen (e.g., wherein such epitope substantially comprises a alpha 2-6 linked NeuAc on a carbohydrate chain such as NeuAcα2→6Gal-R, wherein R comprises the rest of the carbohydrate chain of glycomolecule; e.g., NeuAcα2→6Galβ1→4GlcNAc→as found on a carbohydrate chain of carcinoembryonic antigen (CEA) molecules which can be released or shed by adenocarcinomas);

(d) is capable of inducing a humoral immune response resulting in the production and secretion of anti-shed antigen antibody of IgG subtype (including IgG1 or IgG1 and IgG3); and

(e) can interact with anti-shed antigen antibody capable of binding FcγRI in forming immune complexes, wherein the immune complexes may bind and cross-link FcγRI present on the surface of FcγRI expressing cells.

For example, tumors cells produce a shed antigen which comprises a molecule which is exemplified by mucin and mucin-like molecules. Briefly, mucins are high molecular weight glycoproteins (e.g., greater than about 100 kiloDaltons (kD) in molecular mass). In processes such as transformation (e.g., pre-cancerous) or tumor development, and due to various factors (e.g., the increased production of mucin, lack of availability of glycosyltransferases), tumor cells produce mucin in a form of altered glycosylation (e.g., underglycosylated or incompletely glycosylated; and with a terminal sialic acid) as compared to the same types of cells which are not undergoing such a process. Other examples of shed antigen include mucin-like glycoproteins which are differentially glycosylated and shed or released by tumor cells (e.g., CEA and SSEA-1 antigen); human meconium glycoproteins and submaxillary mucins, normally produced by some mammals (e.g., bovine, ovine, porcine), and which have terminal sTn epitopes (such submaxillary mucins are commercially available). Shed antigen may also comprise a glycolipid having 2 or more 2-6 linked, terminal sialic acids per molecule.

The term “immune complexes” is used herein, for purposes of the specification and claims, to mean immune complexes comprised of shed antigen complexed to anti-shed antigen antibody, wherein the anti-shed antigen antibody comprises an IgG subtype capable of binding to FcγRI primarily via the Fc portion of the antibody (e.g., preferably of IgG1 subtype, or IgG3 subtype, or a combination thereof), and in a more preferred embodiment the anti-shed antigen antibody comprises IgG1 subtype. The term anti-shed antigen antibody is used herein, for purposes of the specification and claims, to mean an antibody having binding specificity for a carbohydrate epitope comprising a terminal 2-6 linked sialic acid found in carbohydrate chains of the shed antigen.

The term “cytokine” is used herein, for purposes of the specification and claims, and with particular reference to FcγRI-expressing cells, to mean a protein (that is not an antibody) that acts as an intercellular mediator, and of which production is induced (and the cytokine may be released) following cross-linking by immune complexes of FcγRI on, and resultant activation of, FcγRI-expressing cells. As apparent to one skilled in the art, the one or more cytokines induced as a result of the activation process will depend on which cell type of FcγRI-expressing cells is activated; and may comprise interleukin, interferon, tumor necrosis factor, chemoattractant, growth factor, adhesion molecule, metalloproteinase, cyclooxygenase (“COX”), degradative (e.g., hydrolytic) enzyme, angiogenic factor, and a combination thereof; as will be more apparent from the following descriptions. In a preferred embodiment, cytokines induced as a result of activation of immune effector cells and of endothelial cells results in production of TNF-α, or IL-1β, or cyclo-oxygenase-2 (COX-2), or a combination thereof Depending on the cell type, other cytokines which may be induced may include one or more of: VEGF, vascular cell adhesion molecule-1 (VCAM-1), monocyte chemoattractant protein-1 (MCP-1), matrix metalloproteinases (“MMPs”; e.g., MMP-2 and/or MMP-9), by cross-linked endothelial cells; IL-1α, IL-6. IL-4, Interferon-gamma (IFN-γ), intracellular adhesion molecule-1 (ICAM-1), MMPs, MCP-1, RANTES, and VEGF by cross-linked microglial or by cross-linked astrocytes or by cross-linked macrophages; and neutrophil primary granule proteases or degradative proteases (e.g., myeloperoxidase, elastase, collagenase, gelatinase, etc.), by cross-linked neutrophils. A preferred cytokine or combination of cytokines may be assayed as a biological response from, cross-linked cells to the to the exclusion of a cytokine or combination of cytokines other than the preferred cytokine or combination of cytokines. As apparent to one skilled in the art, the one or more cytokines may be measured using an immunoassay, or the mRNA (e.g., as a measure of gene activation) for a cytokine may be detected and/or quantitated such as by use of one or more nucleic acid amplification techniques known in the art.

The term “endothelial cells” is used herein, for purposes of the specification and claims, to mean endothelial cells of a type selected from the group consisting of arterial endothelial cells, aortic endothelial cells, vascular endothelial cells (and preferably, umbilical vascular endothelial cells), venule endothelial cells, capillary endothelial cells, sinusoidal endothelial cells, a cell line derived therefrom, ad a combination thereof. A preferred type of endothelial cell may be used as the FcγRI-expressing cells in the assay to the to the exclusion of endothelial cells other than the preferred endothelial cells.

The term “proliferation” is used herein, for purposes of the specification and claims, and with reference to a cell proliferation assay according to the present invention, to mean an increase in any one or more of cell number, rate of cell growth, cell division, and cell survival (e.g., which may manifest as an increase in cell number).

The term “assay” is used herein, for purposes of the specification and claims, to mean an analysis for measuring the biological response that may be induced as a result of the activation of the FcγRI-expressing cells by immune complexes comprising shed antigen and anti-shed antigen antibody of IgG subtype, wherein the analysis may be performed (depending on the biological response to be assayed) by a method which may include, but is not limited to, an immunoassay (e.g., enzyme linked immunoassays, fluorescence-based immunoassays, chemiluminescence immunoassays, Western blots, microarrays, and the like), biochemical analysis (e.g., mass spectrometry, high pressure liquid chromatography, and the like) mRNA levels (e.g., Northern blots, nucleic acid amplification techniques, microarrays, and the like), a cell proliferation assay (e.g., cell counting, or calorimetric measurement such as cellular acid phosphatase or MTT, or flow cytometry), enzyme activity assay (for biological responses in which enzyme activity rather than the amount of enzyme molecule itself is assayed), tumor cell invasion assay, cell movement assays in specialized migration chambers (for chemotactic factors or angiogenic factors), and cell surface or internal molecule expression (flow cytometry, microscopy). A preferred type of assay may be used to the exclusion of a type of assay other than the preferred assay.

The present invention relates to the discovery of a novel mechanism by which a particular and distinct class of immune complexes, immune complexes comprised of shed antigen and anti-shed antigen antibody of the IgG subtype capable of binding FcγRI, induce a biological response. In a more preferred embodiment, the antibody of the IgG subtype comprises antibody of the IgG1 subtype. For example, where the shed antigen comprises shed tumor mucin, anti-sTn antibody can bind to the sTn antigen spaced along a shed mucin molecule. In conditions favoring immune complex formation (versus in antigen excess), these immune complexes have IgG in a number and spacing which are optimal for binding and cross-linking FcγRI (e.g., “immunostimulatory” immune complexes; see, e.g., FIG. 2). Immune complex binding and cross-linking of FcγRI on FcγRI-expressing cells can result in induction-of a biological response that (a) in vivo, may contribute to disease progression, and (b) in vitro, may be assayed using any one of several methods known in the art for assaying the presence of a measurable biological response. In a preferred embodiment of this assay, the antibody used and the FcγRI-expressing cells used should preferably be species-specific (e.g., use of a anti-human shed antigen IgG1 mAb in conjunction with use of FcγRI-expressing human cells; and use of a anti-murine shed antigen IgG1 mAb in conjunction with use of FcγRI-expressing murine cells); however, some cross-species specificity may exist. An in vitro assay provides a simple, rapid, and quantifiable method to assay for (a) inhibitors of immune complex-mediated activation of FcγRI-expressing cells; and (b) for inducers comprising a shed antigen which, when complexed with anti-shed antigen antibody capable of binding FcγRI in forming immunostimulatory immune complexes, can result in immune complex-mediated activation of FcγRI-expressing cells. More particularly, while the assays according to the present invention have several applications, a preferred application is for drug discovery. For example, substances (e.g., peptides, drugs, chemicals, compounds, agents, hormones, cytokines, apatmers, antibodies, expression vectors for expressing a desired protein or nucleic acid molecule) may be screened in the assay according to the present invention to identify of inhibitors of activation of FcγRI-expressing cells by measuring the biological response in the presence of the substance, and comparing it to the biological response measured in the absence of the substance. In the assay, a substance that is an inhibitor inhibits activation so that the measured biological response produced in the presence of the substance is comparatively less (in amount, activity, or number, depending on the biological response measured) than the comparative biological response produced from comparative cross-linked cells in the absence of the substance.

In a preferred embodiment, the substance selected to be screened in the assay according to the present invention comprises a substance of unknown pharmacological activity. For example, combinatorial methods and peptide- or phage-display methods result in a library of substances of unknown pharmacological activity from which selected substances may be screened for pharmacological activity. Thus, a substance of unknown pharmacological activity may be screened in the assay according to the present invention to identify whether or not the substance comprises an inhibitor of activation of FcγRI-expressing cells (a specific pharmacological activity). Where a substance of unknown pharmacological activity is identified by the assay according to the present as an inhibitor of activation of FcγRI-expressing cells, the present invention further comprises the use of that substance as an inhibitor of activation of FcγRI-expressing cells (e.g., either in vitro, as a drug development assay for further characterizing the activity and efficacy of the substance, or in vivo, as a therapeutic). For example, such use of the substance as an inhibitor of activation of FcγRI-expressing cells may comprise administering (e.g., by an appropriate route of administration, which may include, but is not limited to, intravenously, orally, intramuscularly, subcutaneously, intranasally, intraperitoneally, and into lymphatic vessels; and which depends on the particular substance and the health of the individual to be treated), a therapeutically effective amount of the substance to an individual in a method of inhibiting activation of FcγRI-expressing cells in the treated individual. A therapeutically effective amount of the substance comprises an amount sufficient to effect inhibit immune complex-mediated activation of FcγRI-expressing cells in vivo. It will be appreciated by those skilled in the art that the particular dosage, timing, and regimen will depend on such factors as properties inherent to the substance (e.g., clearance, and half life); the health, size, and metabolism of the individual undergoing treatment.

### EXAMPLE 1

In this example, it is important to consider the following concept. Various strains of mice were used as a standard animal model for evaluating whether a humoral immune response against a tumor producing shed antigen (including shed tumor mucin) may be involved in tumor progression (one or more of tumor growth, invasion and metastasis). In tumor bearing mice of B cell competent strains, a similar B cell response (particularly to shed antigens) was observed in lymph nodes regional to a primary tumor as observed in tumor bearing humans. In this example, tested was whether the interruption of the humoral immune response in a tumor bearing animal would affect tumor progression. Twenty C3H mice were injected intrasplenically with 10⁶Met 129 tumor cells. Met 129 cells produce antigen including shed tumor mucin. The injected mice were then divided into three treatment groups. One group of 6 mice was injected with phosphate buffered saline (PBS) at days 5, 7, and 9 following tumor challenge. A second group consisted of 8 mice injected with an irrelevant (not directed against any specific mouse antigen) goat IgG antibody (170 μg per injection) at days 5, 7, and 9 following tumor challenge. A third group consisted of 6 mice injected with goat anti-mouse IgG (170 μg per injection) at days 5, 7, and 9 following tumor challenge. The goat anti-mouse IgG was used to deplete the C3H mice of their B cells, thereby interrupting the production of anti-shed antigen antibody, and therefore preventing the production of immune complexes comprised of shed antigen and anti-shed antigen antibody. At 22 days following tumor challenge, the three groups of mice were analyzed for primary tumor growth in the spleen, metastasis to the liver, and extra-regional metastasis (abdominal lymph nodes). Table 1 shows a comparison of primary tumor growth, and the incidence of liver metastasis (“Liver Met.”) and extra-regional metastasis (“Extra-R Met.”) in the mice treated with PBS (“Control”), mice treated with irrelevant goat IgG (“Goat-IgG”), and mice treated with goat anti-mouse IgG (“Anti-IgG”). Table 1 shows that there is a statistically significant reduction in the incidence of metastasis in the mice in which was inhibited the formation of antibodies against shed tumor antigen, and immune complexes comprised thereof (“Anti-IgG”) as compared to the control group or group receiving irrelevant IgG.

TABLE 1

| Observed | Control | Goat-IgG | Anti-IgG |
|---|---|---|---|
| Tumor | 6 of 6 | 8 of 8 | 6 of 6 |
| Liver Met. | 6 of 6 | 5 of 8 | 0 of 6 |
| Extra-R Met. | 6 of 6 | 6 of 8 | 0 of 6 |

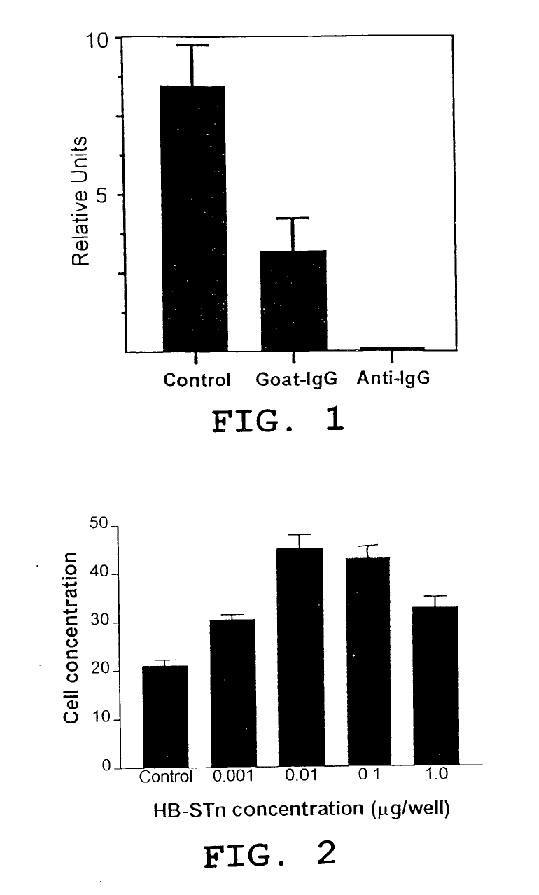

Spleen tumor was scored and compared among the three groups of mice. Treatment of the tumor bearing mice with either goat IgG, or goat anti-mouse IgG had little effect on the growth of the primary tumor in the spleens of the treated animals. In contrast, mice receiving treatment with goat anti-mouse IgG showed a statistically significant reduction in the incidence of liver metastasis as compared to the liver metastasis exhibited by either the control group of mice or the group of mice treated with irrelevant goat IgG (see FIG. 1). However, it was noted that mice receiving treatment with irrelevant goat IgG also showed a statistically significant reduction in the incidence of liver metastasis as compared to the liver metastasis exhibited by the control group (FIG. 1). This latter observation supports the premise that a molecule having binding affinity for FcγRI can effect a reduction in tumor progression.

In summary, the results illustrated in Table 1, and FIG. 1 further support the finding that by inhibiting the formation of immune complexes containing anti-shed antigen IgG antibody, or by blocking FcγRI binding to or cross-linking by immune complexes, in vivo tumor progression can be inhibited.

### EXAMPLE 2

In this example, illustrated: (a) are methods to determine if a cell type is expressing FcγRI; (b) is evidence that nonlymphoid tumor cells can be FcγRI-expressing cells; and (c) are results show that cross-linking of FcγRI by immune complexes may be one signal that induces tumor cell proliferation. Murine mammary tumor cell line Met 129, human colorectal carcinoma cell line SW620, and human breast carcinoma cell line T-47D were tested for Fc receptor expression: FcγRI (CD64), FcγR!I (CD32), and FcγRIII (CD16). T-47D tumor cells were cultured adherently (37° C+5% CO₂) in serum-free tissue culture medium; SW620 tumor cells were cultured in suspension (37° C. minus CO₂) in serum-free tissue culture medium; and Met 129 tumor cells were grown in vivo in mice using methods already described herein. All three of these cell lines are high mucin producers. Depending on the cell culture conditions, the cultured cells were collected from suspension or scraped from adherent culture after washing twice with PBS. Dispersed cells were centrifuged at 1200 rpm for 10 minutes, and the subsequent cell pellet was resuspended in PBS without calcium and magnesium. Cells were counted, and cell viability was checked using trypan blue exclusion dye. After determining the cell count, cells were aliquoted into 1.5 ml microfuge tubes at a concentration of one million cells per tube. Cells were pelleted by centrifugation at 3000 rpm for 3 minutes. The supernatant was removed, and pellets were resuspended in 40 μl of antibody suspension according to the schedule illustrated in Table 2.

TABLE 2

| Fluorochrome | Tube # | Antibody |
|---|---|---|
| none | 1 | none-cells unstained |
| PE | 2 | isotype control (mouse IgG1) |
| PE | 3 | isotype control (mouse IgG2a) |
| Pc5 | 4 | mouse anti-human CD16 (IgG1) |
| PE | 5 | mouse anti-human CD32 (IgG2a) |
| FITC | 6 | mouse anti-human CD64 (IgG1) |

PE is phycoerythrin, FITC is fluoroscein-isothiocyanate, and Pc5 is phycoerythrin-cyanine 5.

The cells were incubated with antibody for 30 minutes on ice. After staining, the cells were pelleted by centrifugation at 3000 rpm for 3 minutes, and the supernatants were removed. The cell pellets were washed by resuspension in 100 μl PBS, followed by recentrifugation. The supernatants were then removed, and the pellets were resuspended in 250-350 μl of PBS for analysis by flow cytometry. The samples were analyzed on a flow cytometer by forward scatter, side scatter, and by excitation using an argon laser at 488 nm. The fluorescent signals were collected through a 530 nm band pass filter for the FITC emissions, a 585 nm band pass filter for the PE emissions, and a 670 nm band pass filter for the Pc5 emissions. As shown in Table 3, only FcγRI (CD64) was significantly expressed by the three different tumor cell lines (% FcR is percentage of gated cells positive for that receptor, after correction for non-specific binding events by determining by non-specific binding by the isotype control antibody). While FcγRII expression by tumor cells has been described previously, the present inventors are unaware of any published reports on expression of FcγRI by nonlymphoid tumor cells.

TABLE 3

| Receptor | SW620 | T-47D | Met 129 |
|---|---|---|---|
| CD16 | 0.4% | 0% | 0% |
| CD32 | 1.4% | 1.5% | 0% |
| CD64 | 14.9% | 33.1% | 33.9% |

FcγRI expression on T47-D cells and SW620 cells was also confirmed by nucleic acid amplification involving reverse transcription of mRNA and amplifying the resultant cDNA using standard techniques. Briefly, total RNA isolated from the tumor cells was reversed transcribed to cDNA using a commercial reverse transcriptase kit. FcγRI-specific primers SEQ ID NO:1 and SEQ ID NO:2 were combined with the cDNA in a polymerase chain reaction performed with the following conditions: 95° C. for 5 minutes, 55° C. for 3 minutes, 72° C. for 1 minute, and a 15 minute extension at 72° C. The amplified fragments were separated by agarose gel electrophoresis, and bands were purified for DNA sequencing. By sequence analysis, the FcγRI expressed by T47-D cells and SW620 cells may comprised FcγRIA, and an alternatively spliced transcript corresponding in sequence to FcγRIB. FcγRIB contains exons EC1 and EC2, lacks exon EC3, and contains the transmembrane domain. It has published that cells expressing FcγRIB can bind immune complexes comprised of human IgG (preferentially binding the Fc portion of the IgG1 subtype, and IgG3 subtype).

To illustrate one preferred embodiment of the assay according to the present invention, assayed was a measurable biological response produced in vitro as a result of activation of FcγRI-expressing cells comprising tumor cells by immune complexes comprising shed antigen complexed to anti-shed antigen IgG antibody. In this illustration, high mucin producing cell lines murine Met 129, human T-47D breast cancer cells, and human SW620 colon carcinoma cells were each separately treated with anti-sTn antibody (IgG1 monoclonal antibody; “mAb”) in in vitro culture. Thus, addition of the anti-sTn mAb would complex to sTn-containing tumor mucin shed by the tumor cells in producing soluble immune complexes. At approximately 16 hours prior to initiating assay, each adherent cultured cell line was trypsinized and reseeded at a low concentration in serum-free tissue culture medium (medium which is supplemented with growth factors, but does not include serum as a component; commercially available; e.g., ULTRA CHO). After incubation, cells were scraped and centrifuged at 1200 rpm for 10 minutes. A cell count was performed, and viability was assessed using trypan blue exclusion. Cell concentration was adjusted to 1000 cells/90 μl in the serum-free tissue culture medium. Monoclonal mouse anti-sTn antigen antibody (IgG1; DAKO-HB-STn1), supplied at a concentration of 90 μg antibody/ml, was dialyzed in sterile PBS without Ca/Mg using a 2000 dalton molecular weight cutoff for 20-24 hours to remove sodium azide. After dialysis, the following dilutions of antibody were prepared in the serum-free tissue culture medium: 1.0 μg/10 μl; 0.1 μg/10 μl; 0.01 μg/10 μl; and 0.001 μg/10 μl. The cells were aliquoted into 96 well plates at 1000 cells/90 μl per well for a total of 30 wells per cell line. Immediately following seeding of cells, 10 μl of the respective diluted and dialyzed anti-sTn antibody was added to each well for a total of 6 wells per antibody dilution. As a control containing no antibody, 10 μl of the serum-free tissue culture medium was added to each of 6 wells. 96-well plates were incubated for approximately 72 hours at 37° C. in 5% CO₂. After 72 hours of incubation, proliferation was assessed by counting adherent cells in each well (e.g., using a microscope). As an alternative, after an appropriate incubation time in the presence of the immune complexes, the culture medium is removed. MTT (3-[4,5-Dimethylthiazol-2-yl]-2,5-diphenyl-tetrazolium bromide; thiazolyl blue) dye may then be added to the cells in each well (final concentration of 0.5 mg/ml); the plate is incubated for 4 hours; the dye is solubilized with 0.1N HCl in absolute isopropanol; and cell proliferation is represented for each well as the absorbance measured at 570 nm minus the absorbance at 690 nm.

FIG. 2 is a bar graph showing the number of T-47D tumor cells plotted in relation to various concentrations of anti-sTn antibody. Similar growth induction curves were observed for Met 129 and SW620 carcinoma cell lines, as well. A statistically significant increase in tumor cell growth was evident, particularly at antibody concentrations of 0.01 μg per well and 0.1 μg per well. The bell-shaped curve illustrated in FIG. 2 is evidence that it is immune complexes, formed between the added anti-sTn IgG1 antibody and shed antigen (e.g., shed tumor mucin bearing sTn antigen), which interact directly with tumor cells to promote tumor cell proliferation. In that regard, an antibody concentration of 0.001 μg per well may represent sTn antigen excess, and an antibody concentration of 1 μg per well may represent antibody excess. Optimal immune complex formation, occurring at antibody concentrations of 0.01 μg per well and 0.1 μg per well, would therefore be more effective (“immunostimulatory”) in inducing tumor cell proliferation. Additionally, the results show that the repeated carbohydrate epitope of the shed antigen secreted by tumor cells, and IgG antibody bound thereto which is capable of binding FcγRI, can provide the required number and spacing in an immune complex for cross-linking FcγRI on FcγRI-expressing cells (formation of “immunostimulatory immune complexes”).

To confirm that tumor cell proliferation is a measurable biological response from cross-linked, FcγRI-expressing tumor cells, the above-described assay was performed using metastatic melanoma cells, B16F10 melanoma cells. We have found that these melanoma cells also express FcγRI, but are not high producers of shed antigen comprising sTn-expressing tumor mucin-1. The melanoma cells were treated in vitro culture with either anti-sTn antibody, or with anti-sTn antibody and tissue culture fluid containing shed antigen comprising tumor mucin. Briefly, to one set of wells, 10 μl of the tissue culture medium was removed from each well at 24 hour intervals and replaced with fresh tissue culture medium (thus, not receiving exogenous tumor mucin, “without mucin”). To another set of wells, 10 μl of the tissue culture medium was removed from each well at 24 hour intervals and replaced with cell-free tissue culture supernatant from cultured T-47D cells which supernatant contains shed tumor mucin (thus receiving exogenous shed antigen, “with mucin”). After 72 hours of co-incubation, proliferation was assessed by counting adherent cells in each well. FIG. 3 is a bar graph showing the relative proliferation (normalized data) of B16F10 melanoma cells plotted in relation to the various concentrations anti-sTn antibody without added mucin, comparative to B16F10 melanoma cells plotted in relation to the various concentrations of anti-sTn antibody with added mucin. Observed is a statistically significant increase in B16F10 melanoma cell proliferation in the presence of immune complexes containing anti-shed antigen antibody and shed antigen (FIG. 3: “with mucin”), particularly at antibody concentrations of 0.01 μg per well and 0.1 μg per well, as compared to the growth of B16F10 melanoma cells co-cultured only in the presence of anti-shed antigen antibody (FIG. 3: “without mucin”). As a general guideline, the time period in which the biological response (e.g., cell proliferation) may be measured for in the assay comprises a time period in the range of from about 2 hours to about 72 hours. As-apparent to one skilled in the art, that time period may vary depending on the number of cells used in the assay, the nature and amount of shed antigen present in the assay, and the nature and amount of anti-shed antigen antibody added to the assay, and the nature of the biological response being measured.

Taken together, these results support the assay according to the present invention comprising assaying for a measurable biological response from FcγRI-expressing cells that have been bound and cross-linked (with resultant activation) by immune complexes comprising shed antigen and anti-shed antigen IgG antibody capable of binding FcγRI. In this illustrated, preferred embodiment, the FcγRI-expressing cells comprise tumor cells, and the measurable biological response comprises an induction in tumor cell proliferation.

In another illustration of this embodiment, a substance was assayed for its ability, if any, to act as an inhibitor of the activation of FcγRI-expressing tumor cells; i.e., to inhibit the measurable biological response comprising an induction in tumor cell proliferation. As previously described herein, FcγRIB comprises a predominant isoform of FcγRI produced by FcγRI-expressing tumor cells. FcγRIB has low affinity for binding of monomeric IgG1, and high affinity for immune complex binding. In this illustrative embodiment, the substance assayed was a murine anti-human CD32 (anti-FcγRII) mAb which has the ability to block immune complexes from binding to human FcγRII, but lacks detectable ability to block immune complexes from binding to human FcγRI. As described above, T-47D cells were seeded 1000 cells per well in a 96 well plate. To one group of cells was added 100 μl of serum-free tissue culture medium containing 10 pg/ml of anti-sTn antigen antibody (IgG1), an amount of antibody sufficient to form immunostimulatory immune complexes that can activate FcγRI-expressing tumor cells to induce tumor cell proliferation. To another group of cells was added 100 μl of serum-free tissue culture medium containing 10 pg/ml of anti-sTn antigen antibody and either 1 pg/ml, or 10 pg/ml, or 100 pg/ml of anti-human FcγRII mAb (IgG1). To a third group of cells was added 100 μl of serum-free tissue culture medium containing 10 pg/ml of anti-sTn antigen antibody and either 1 pg/ml, or 10 pg/ml, or 100 pg/ml of an isotype control mAb (a mAb of the same subtype (here, IgG1) which does not have any detectable binding specificity for tumor cell antigens in the assay). The plate was then incubated for at least 2 hours (and more preferably, 24 hours) at 37° C., after which time the culture medium from each well was removed (the culture medium may be further analyzed for induction of a measurable biological response as well, if desired). MTT was then added (final concentration of 0.5 mg/ml) to the cells in each well, and the plate was then incubated for 4 hours at 37° C., followed by solubilizing the dye with 0.1N HCl in absolute isopropanol, and reading of the absorbance at 570 nm minus the absorbance at 690 nm. The results showed that neither the anti-FcγRII mAb (at any of the concentrations 1 pg/ml, or 10 pg/ml, or 100 pg/ml) nor the monomeric isotype control mAb (at any of the concentrations 1 pg/ml, or 10 pg/ml, or 100 pg/ml) inhibited the measurable biological response comprising an induction in tumor cell proliferation. Neither of the substances (anti-FcγRII mAb or the monomeric IgG), assayed for their ability to be inhibitors of activation of FcγRI-expressing tumor cells, comprise inhibitors of activation. However, the failure of the anti-FcγRII mAb to inhibit immune complex activation of the FcγRI further demonstrates that FcγRIB plays a predominant role in the binding of immune complexes to the tumor cells.

### EXAMPLE 3

In this example, illustrated is another preferred embodiment of the assay according to the present invention. In this example, assayed was a measurable biological response produced in vitro as a result of activation of FcγRI-expressing cells comprising tumor cells by immune complexes comprising shed antigen complexed to anti-shed antigen IgG antibody capable of binding FcγRI; wherein the biological response was an induction of shed antigen (e.g., shed tumor mucin) production. An assay in which assayed for is a biological response comprising an increase in shed tumor mucin (Muc-1; “mucin”) production by cross-linked FcγRI-expressing tumor cells in vitro has clinical applications for (a) identifying tumor cells isolated from a patient to determine if the isolated tumor cells exhibit this measurable biological response, or (b) for screening for inhibitors of activation which results in this biological response. More specifically, as known to those skilled in the art, the amount of mucin produced by adenocarcinomas in vivo (a) is associated with the metastatic potential of the tumor, (b) may be used as a marker of progression and metastasis, and (c) may be used an prognostic indicator (e.g., production of a high amount of mucin by adenocarcinomas correlates with a poorer prognosis for the patient than the prognosis associated with an adenocarcinoma of the same tissue type which produces a lower amount). Thus, by identifying a mechanism by which shed tumor mucin production is induced, and by providing an in vitro assay in which this biological response is measurable, a clinician is provided with an assay for determining if a patient's tumor is one that can be activated to produce a high amount of shed tumor mucin. If the tumor cells do demonstrate this ability in the in vitro assay, the clinician may choose to use such a finding in decisions related to treatment of the patient (i.e., may treat the tumor with more aggressive anti-tumor therapy). Likewise, by identifying an inhibitor of this biological response by using the in vitro assay, such an inhibitor may then be considered as a possible therapeutic agent in the treatment of a patient having tumor which demonstrates this measurable biological response in vitro.

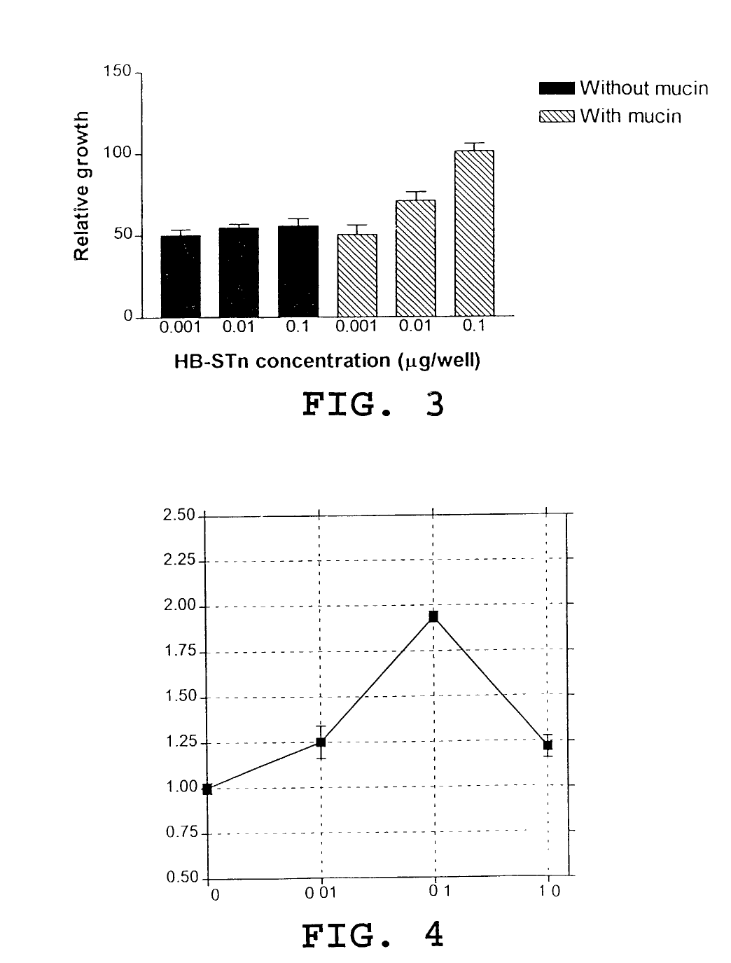

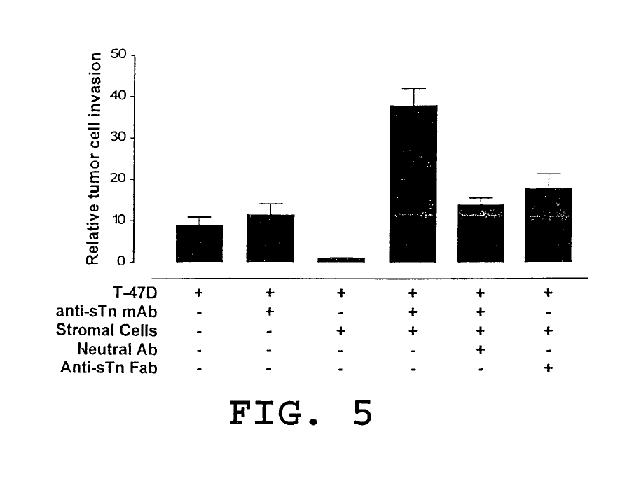

In this experiment, plated in each well of a 24 well plate was 100,000 T-47D breast carcinoma cells. The cells were cultured in standard tissue culture medium for 24 hours at 37° C. in 5% CO₂, after which time the medium was removed. To individual groups of wells was added 200 μl of tissue culture medium containing one of the following dilutions of anti-sTn IgG1 mAb: 1 ng/ml, 0.1 ng/ml, 0.01 ng/ml, and 0 ng/ml. The cells were cultured for 2 hours, after which time the cells were washed three times with fresh tissue culture medium. To the cells was added 200pl of the medium alone, and the cells were cultured for an additional 2 hours. The medium from each culture was then collected separately from each well, centrifuged, and the individual culture supernatants were assayed for mucin production by measuring the amount of sTn epitope detected in an enzyme-linked immunosorbent assay (ELISA). The cells in each well were assayed for proliferation by the addition of MTT (as previously described herein). FIG. 4 shows the normalized data for this experiment, in which the ratio of the amount of mucin to the amount of cell proliferation is plotted versus the concentration of anti-sTn IgG1 mAb added. As shown in FIG. 4, at a concentration of 0.1 ng/ml of anti-sTn IgG1 mAb, and after 2 hours of incubation with cells producing shed tumor mucin in the culture medium, the shed tumor mucin production increased approximately 200 fold over the comparative control cells to which no anti-sTn IgG1 mAb was added. In a parallel assay, treatment of T-47D breast carcinoma cells with an isotype control antibody (a mAb of the same immunoglobulin subtype (here, IgG1) which does not have any known binding specificity for components in the assay (e.g., here, shed tumor antigen)) did not induce a detectable increase in mucin production as compared to the comparative control cells to which no IgG1 mAb was added.

The results illustrated in FIG. 4 indicate that besides being a mechanism which may activate FcγRI-expressing tumor cells to induce tumor growth, immune complexes (formed between anti-shed tumor antigen antibody capable of binding FcγRI, and shed tumor antigen) may also interact directly with FcγRI-expressing tumor cells to induce an increase in the production of shed tumor antigen, or may produce a measurable biological response other than induction of shed antigen production and cell proliferation.

### EXAMPLE 4

Illustrated in this example is that immune complexes comprising shed antigen and anti-shed antigen mAb capable of binding FcγRI may also bind to and cross-link FcγRI on FcγRI-expressing immune effector cells (one or more cell types comprising neutrophils, macrophages) to promote tumor progression. Additionally, illustrated is an embodiment of the assay according to the present invention in which a measurable biological response is measured using a tumor cell invasion assay. To illustrate this embodiment, granulocytes (polymorphonuclear cells) were harvested by positive selection from peripheral blood, and macrophages were harvested by peritoneal wash, from C3H-SCID beige mice in order to avoid contaminating mouse NK cells, natural Abs, or any immune recognition by mouse T or B lymphocytes. In this in vitro tumor cell invasion assay, used were mucin-secreting human tumor cell line T-47D, a Boyden chamber, and a commercially available basement membrane matrix preparation (“matrix”) which solidifies into a semisolid matrix. In this assay, tested was the ability of tumor cells (2×10⁴ cells) to migrate through the matrix (layered into a 6.5 mm well insert containing a polycarbonate membrane having a 3.0 μm pore size) in the following conditions: matrix alone; matrix containing stromal cells (2×10⁵ cells, granulocytes and macrophages combined); matrix in the presence of anti-shed tumor antigen antibody (anti-sTn mAb; IgG1; 0.06 μg); matrix containing stromal cells in the presence of anti-sTn mAb (0.06 μg); and matrix containing stromal cells in the presence of Fab fragments (0.06 μg) of the anti-sTn mAb. The plates were incubated at 37° C. in 5% CO₂, and fresh tissue culture medium (with or without antibody/antibody fragment, depending on which of the above conditions were assayed) was substituted every 24 hours. Invasion was measured by counting the number of tumor cells per well which migrated to the bottom of the chamber after 48 to 72 hours.

FIG. 5 shows that the maximum invasion through the matrix was observed when the shed tumor antigen (mucin)-secreting tumor cells were incubated in the presence of stromal cells and either of two anti-sTn IgG1 mAb tested (“anti-sTn mAb”) as compared to tumor cells alone, or stromal cells and tumor cells, or anti-sTn mAb and tumor cells, or stromal cells and tumor cells in the presence of Fab fragments of the anti-sTn mAb (“Anti-sTn Fab”). These experiments are further evidence that shed tumor antigen secreted by tumor cells can interact with anti-shed antigen antibody (e.g., of IgG1 subtype) in forming complexes that can bind, cross-link and activate FcγRI-expressing cells, such as granulocytes and macrophages, to secrete enzymes and/or factors (e.g., one or more of tissue degradative enzymes, cytokines, oxygen free radicals) that promote tumor progression. The involvement of immune complexes, as opposed to the action of antibody alone, was confirmed by using a tumor cell type which did not produce detectable amounts of sTn-containing shed tumor antigen (e.g., melanoma cells); i.e., when such tumor cells were incubated in the presence of stromal cells and anti-sTn mAb, there was no increase in tumor invasion as compared to the control values. It is important to note that if tumor cell invasion is comparatively assayed using the anti-sTn mAb with either granulocytes or macrophages, no significant induction of tumor cell invasion is observed (e.g., this induction appears to require the presence of both granulocytes and macrophages).

Illustrated is an embodiment according to the present invention wherein the assay is used to screen substances for their ability, if any, to inhibit activation of FcγRI-expressing cells by shed antigen-containing immune complexes. In this embodiment, tested was the ability of a monomeric IgG1 antibody (murine anti-rabbit LDL receptor mAb not known to any binding specificity for any component in this assay) to inhibit activation of FcγRI-expressing cells by immune complexes comprising shed tumor antigen and anti-shed tumor antigen antibody. By performing the assay as described above, including tumor cells, stromal cells, anti-sTn mAb (0.06 μg) and the monomeric IgG1 antibody (0.06 μg; FIG. 5, “Neutral Ab”), observed was a reduction in tumor cell invasion of approximately two fold as compared to the maximum tumor cell invasion observed when the shed tumor antigen-secreting tumor cells were incubated in the presence of stromal cells and anti-sTn IgG1 mAb. Thus, using the assay according to the present invention, identified was an inhibitor, monomeric IgG1, of activation of FcγRI-expressing immune effector cells to produce a measurable biological response comprising promotion of in vitro tumor invasion.

In another example of using the assay according to the present invention, in the form of a tumor cell invasion assay, substances were screening for identifying inhibitors of activation. In this example, assayed were inhibitors of factors released by neutrophils. More specifically, and with the proper assay controls, the tumor cell invasion assay was performed as described above using tumor cells, stromal cells, anti-stn IgG1 mAb (previously dialyzed to remove azide), in the presence of sodium azide (0.001% per well) as an inhibitor of myeloperoxidase, or in the presence of methoxysuccinyl-alanyl-alanyl-prolyl-valine-chloromethylketone (“MAAPVC”; 0.1 μg/well) as an inhibitor of granulocyte elastase. The results showed that there was an significant (e.g., approximate 5 fold) reduction in the tumor cell migration in the presence of azide as compared to the comparative assay performed in the absence of azide. Thus, the assay identified azide as an inhibitor of activation of FcγRI-expressing cells comprising neutrophils, wherein the inhibitor inhibits a biological response (e.g., myeloperoxidase activity) induced by activation. The results also show that there was an approximate 5 fold reduction in the tumor cell migration in the presence of MAAPVC as compared to the comparative assay performed in the absence of MAAPVC. Thus, the assay identified MAAPVC as an inhibitor of activation of FcγRI-expressing cells comprising neutrophils, wherein the inhibitor inhibits a measurable biological response (e.g., elastase activity) induced by activation. Additionally, these inhibitors, azide and MAAPVC, are inhibitors of a measurable biological response comprising tumor cell migration in the presence of FcγRI-expressing cells comprising neutrophils and tumor cells.

### EXAMPLE 5

Angiogenesis, new blood vessel formation, is a key process in tumor growth and metastasis; and represents a focus of the development of new anticancer therapies. Typically, new blood vessel formation occurs when endothelial cells from the wall of a small blood vessel are activated to secrete enzymes to degrade the surrounding extracellular matrix allowing the endothelial cells to invade through the matrix, and to proliferate in providing a string of dividing endothelial cells which create the new blood vessel. The proteins which are known to induce endothelial cell proliferation include epidermal growth factor, angiogenin, estrogen, fibroblast growth factors (acidic and basic), and VEGF. In developing the assays according to the present invention, it was discovered that endothelial cells can produce functionally significant and detectable amounts of FcγRI; and that immune complexes can bind and cross-link FcγRI on FcγRI-expressing endothelial cells which may then initiate changes in endothelial cell growth and promotion of angiogenesis (based on both in vitro and in vivo findings). Hence, a novel angiogenic factor comprises immune complexes comprised of shed antigen and anti-shed antigen antibody of IgG subtype capable of binding FcγRI. Such immune complexes can induce endothelial cell proliferation and secretion of additional angiogenic factors which may promote disease progression (e.g., such as tumor progression). In that regard, endothelial cell proliferation is necessary for angiogenesis, and angiogenesis is required for growth and progression of solid, nonlymphoid tumors. In a preferred embodiment, the FcγRI-expressing endothelial cells may include one or more of capillary endothelial cells (e.g., type 1 endothelial cells, or type 2 endothelial cells; Cardier and Barbera-Guillem, 1997, Hepatology, 26:165-175), human umbilical vascular (or vein) endothelial cells (HUVEC), or endotheliomas.

In one illustrative example, the FcγRI-expressing endothelial cells comprised approximately 100,000 cells of either type 1 endothelial cells or type 2 endothelial cells which were added per well in a 24 well plate. Supernatant from a culture of 24,000 cells per well of shed tumor antigen-secreting nonlymphoid tumor cells (e.g., shed tumor mucin-secreting T-47D cells) and 0.06 μg of anti-sTn mAb (IgG1 subtype) were added to the endothelial cells in each well, and the endothelial cells were cultured for various times between 24 hours and 72 hours at 37° C. in 5% CO₂. After a 24 hour period, a measurable biological response was evident by the at least a 50% increase in the number of endothelial cell number in wells in which both the anti-shed tumor antibody and shed tumor antigen were added, as compared to the comparative control wells in which culture supernatant containing shed tumor antigen was added but in which anti-sTn mAb was not added. These results indicate that immune complexes comprising anti-shed antigen IgG mAb and shed antigen may bind to and cross-link FcγRI on FcγRI-expressing endothelial cells in activating the cells to induce endothelial cell proliferation.

In another illustrative example, the immune complexes comprised bovine submaxillary mucin (“BSM”; commercially obtained) and anti-sTn mAb (clone B72.3, IgG1). Plated per well of a 96 well plate were 10,000 type 1 endothelial cells in tissue culture medium, and the cells were incubated overnight. After the incubation, the culture medium was removed, and: to one group of cells was added 100 μl of fresh tissue culture medium containing 0.06 μg of an isotype (IgG1) mAb (9D9, previously described herein); to another group of cells was added 100 μl of fresh tissue culture medium containing 0.75 ng of BSM; to a third group of wells was added 100 μl of fresh tissue culture medium containing 0.06 μg of anti-sTn mAb and 0.75 ng of BSM; and to a control group of wells was added 100 μl of fresh tissue culture medium alone. After a two hour incubation period, the supernatants were collected from each well and then analyzed in a commercial ELISA kit for determining VEGF concentration. The comparative wells containing either tissue culture medium alone, or BSM alone, or the isotype control mAb (9D9) showed an average basal level of VEGF production of around 2.5 to 3.2 pg/ml. In contrast, the wells containing immune complexes comprising anti-sTn mAb and BSM showed an average level of VEGF production of over 5 pg/ml. Thus, there was a statistically significant induction of VEGF production (a measurable biological response) by FcγRI-expressing endothelial cells activated by immune complexes comprising shed antigen and anti-shed antigen IgG antibody. Both VEGF and cell proliferation can be measured from the same assay by including MTT in the above-described assay after the supernatants are collected.

Taken together, immune complexes comprising shed antigen and anti-shed antigen IgG antibody capable of binding FcγRI can activate FcγRI-expressing endothelial cells to result in a measurable biological response comprising either induction of VEGF production, or induction of cell proliferation, or a combination thereof. Further, this assay may be used to induce a measurable biological response (e.g., as previously described herein, an induction of cytokines other than VEGF production) other than an induction of VEGF production or induction of cell proliferation. Thus, this assay has applications for screening for inhibitors of such activation. Presently, there are about 20 angiogenesis inhibitors which may be classified by the following groupings: those that inhibit endothelial cell growth (e.g., IL-12, Platelet factor-4); those that bind to and block VEGF or the VEGF receptor (e.g., mAb to VEGF, SU5416); those that inhibit the release of VEGF (e.g., IFN-α); those that interrupt the function of dividing endothelial cells (e.g., TNP-470); and those which work by a yet to be defined mechanism (e.g., suramin, thalidomide). The novel assay according to the present invention may be used for screening these substances known to be angiogenesis inhibitors, or substances which may be classified by the above-described groupings, as well as other substances (including compounds, drugs, agents, conjugates, organic molecules, and biomolecules) desired to be tested for their ability, if any, to inhibit a measurable biological response (e.g., induction of one or more of VEGF production, endothelial cell proliferation, TNF-α production, IL-1β production, COX-2 production, VCAM-1 production, MCP-1 production, MMP production) by FcγRI-expressing endothelial cells activated by immune complexes comprising shed antigen and anti-shed antigen antibody of IgG subtype capable of binding FcγRI. In one embodiment of performing the assay, immune complexes and the FcγRI-expressing cells are incubated together for sufficient time (e.g., preferably a time period in the range of from about 2 hours to about 72 hours) to allow the immune complexes to contact, bind, and cross-link FcγRI in activating the cross-linked cells to induce of one or more measurable biological responses; detecting the one or more measurable biological responses; and comparing the one or more measured biological responses to comparative (of the same type) one or more biological responses in a comparative assay (e.g. performed in essentially the same conditions) which further comprise the substance being screened as an inhibitor of activation (e.g., the substance is incubated together with the immune complexes and the FcγRI-expressing endothelial cells). Thus, the value of the comparative measurable biological response from the comparative assay performed in the absence of the substance may be compared to the value of the measurable biological response from the assay performed in the presence of the substance. A reduction in the value of the measurable biological response (e.g., VEGF production) determined from the assay in the presence of the substance, as compared to the value of the comparative measurable biological response (induction of VEGF production) determined from the comparative assay in the absence of the substance may be an indicator that the substance may be an inhibitor of activation. Further, in continuing with an example wherein the measurable biological response is VEGF production, the inhibitor may also be considered as an inhibitor of VEGF production. As will be apparent to one skilled in the art, the amount of the substance to be screened for inhibition of activation in the assay depends on factors including, but not limited to, the nature of the screening composition, the number of cells in the assay, the amount of the mixture of shed antigen and anti-shed antigen antibody added, and the assay format. As is apparent to one skilled in the art, typically a serial dilution of the substance being screened may be added to various individual wells containing the immune complexes and FcγRI-expressing cells in the assay.

For example, typically the addition of shed antigen and anti-shed antigen antibody of IgG1 subtype capable of binding FcγRI to the FcγRI-expressing endothelial cells can result in a measurable biological response comprising an increase (induction) of endothelial cell proliferation from about 20% to about 50% or greater as compared to a comparative controls; e.g., containing approximately the same starting number of FcγRI-expressing endothelial cells treated with either shed antigen only, or an isotype control antibody only, or tissue culture medium only. By treating the cells (either by pre-incubating the cells or added simultaneously, or adding subsequently, in relation to the addition of the mixture containing the immune complexes) with a substance to be screened as an inhibitor of activation, analyzed is any effect of the substance in relation to inhibiting the induction of endothelial cell proliferation observed when the FcγRI-expressing endothelial cells are activated by the immune complexes (in the absence of the substance). In a preferred embodiment, a substance that is an inhibitor of activation reduces the measurable biological response (e.g., in this example, the induced endothelial cell proliferation) by at least 20%; and in a more preferred embodiment, a substance that is an inhibitor of activation reduces the measurable biological response (e.g., in this example, the induced endothelial cell proliferation) by at least 50%; and in a more preferred embodiment, a substance that is an inhibitor of activation reduces the measurable biological response (e.g., in this example, the induced endothelial cell proliferation) by at least 90%. The substance to be screened as an inhibitor in the assay may also be added to the FcγRI-expressing cells, in the absence of immune complexes comprising shed antigen and anti-shed antigen IgG antibody, in similar conditions so as to determine whether or not such substance may, by itself, induce a measurable biological response.

### EXAMPLE 6

In this example, illustrated is another preferred embodiment of the assay according to the present invention. In this example, assayed was a measurable biological response produced in vitro as a result of activation of FcγRI-expressing immune effector cells comprising macrophages by immune complexes comprising shed antigen complexed to anti-shed antigen IgG antibody capable of binding FcγRI; wherein the biological response was an induction of cytokine production. In illustrations of this example, macrophages alone, a combination of macrophages and tumor cells, and a combination of macrophages and tumor cells and neutrophils, comprised the FcγRI-expressing cells which were assayed for a measurable biological response as a result of activation by immune complexes comprising BSM and anti-sTn mAb (clone B72.3, IgG1). Briefly, the FcγRI-expressing cells comprised: murine polymorphonuclear cells (“neutrophils”), isolated from the peripheral blood of SCID mice; murine macrophage cell line MH-S, obtained from the American Type Culture Collection; and human breast adenocarcinoma cell line T-47D. 96 well plates were seeded in tissue culture medium with the 10,000 cells/well of the appropriate cell type or combination of cell types (e.g., 5,000 macrophage cells and 5,000 tumor cells), and incubated at 37° C. with 5% CO₂ in culture overnight. The following day the culture medium was removed, and per well was added either 100 μl of fresh tissue culture medium alone, 100 μl of fresh tissue culture medium containing anti-sTn mAb (0.06 μg), or 100 μl of fresh tissue culture medium containing of anti-sTn mAb (0.06 μg) and BSM (0.75 ng). Murine cytokine concentrations were determined using commercially available ELISA kits with standards, as well as appropriate controls. The assay results for cytokine concentrations (listed in pg/ml), with respect to the various cell types and components assayed (“PMN” for neutrophils, “TC” for tumor cells, Mφ for macrophage, “IC” for immune complexes, “mAb” for anti-sTn mAb) are shown in Table 4.

TABLE 4

| Assayed | IL-4 | IL-6 | IFN-γ | IL-1β |
|---|---|---|---|---|
| Mφ | 0.6 | 61.5 | 1.1 | 2.0 |
| Mφ + mAb | 0.6 | 68.7 | 1.2 | 1.8 |
| Mφ + IC | 1.5 | 103.8 | 5.3 | 2.5 |
| Mφ + TC | 0 | 304.3 | 0.8 | — |
| Mφ + TC + IC | 1.3 | 400.5 | 4.0 | — |
| Mφ + TC + PMN | 0 | — | 0.3 | 17.3 |
| Mφ + TC + PMN + IC | 0.6 | — | 4.1 | 22.8 |

As shown in Table 4, macrophages activated by the immune complexes showed an induction comprising a significant increase (e.g., a two-fold increase) in the amount of secreted IL-4 compared to macrophages incubated with anti-sTn mAb in a comparative assay, or macrophages incubated with tissue culture medium alone in a comparative assay. Likewise, macrophages in the presence of tumor cells, and activated by the immune complexes, showed an induction comprising a significant increase in the amount of secreted IL-4 compared to macrophages incubated with tumor cells only in a comparative. Additionally, macrophages in the presence of tumor cells and neutrophils, and activated by the immune complexes, showed an induction comprising a significant increase in the amount of secreted IL-4 compared to macrophages incubated with tumor cells and neutrophils only in a comparative assay. Thus, there was a statistically significant induction of IL-4 production (a measurable biological response) by FcγRI-expressing macrophages (alone or in combination with tumor cells, or in combination with tumor cells and neutrophils) activated by immune complexes comprising shed antigen and anti-shed antigen IgG antibody capable of binding FcγRI.

As shown in Table 4, macrophages activated by the immune complexes showed an induction comprising a significant (e.g., approximately 1.5-fold) increase in the amount of secreted IL-6 compared to macrophages incubated with anti-sTn mAb, or macrophages incubated with tissue culture medium alone. Likewise, macrophages in the presence of tumor cells, and activated by the immune complexes, showed an induction comprising a significant increase in the amount of secreted IL-6 compared to macrophages incubated with tumor cells only. Thus, there was a statistically significant induction of IL-6 production (a measurable biological response) by FcγRI-expressing macrophages (alone or in combination with tumor cells) activated by immune complexes comprising shed antigen and anti-shed antigen IgG antibody capable of binding FcγRI.

As shown in Table 4, macrophages activated by the immune complexes showed an induction comprising a significant (e.g., at least a 4-fold) increase in the amount of secreted IFN-γ compared to macrophages incubated with anti-sTn mAb, or macrophages incubated with tissue culture medium alone. Likewise, macrophages in the presence of tumor cells, and activated by the immune complexes, showed an induction comprising a similar significant increase in the amount of secreted IFN-γ compared to macrophages incubated with tumor cells only. Additionally, macrophages in the presence of tumor cells and neutrophils, and activated by the immune complexes, showed an induction comprising a significant increase in the amount of secreted IFN-γ compared to macrophages incubated with tumor cells and neutrophils only. Thus, there was a statistically significant induction of IFN-γ production (a measurable biological response) by FcγRI-expressing macrophages (alone or in combination with tumor cells, or tumor cells and neutrophils) activated by immune complexes comprising shed antigen and anti-shed antigen IgG antibody capable of binding FcγRI.

As shown in Table 4, macrophages activated by the immune complexes showed an induction comprising a significant increase in the amount of secreted IL-1β compared to macrophages incubated with anti-sTn mAb, or macrophages incubated with tissue culture medium alone. Likewise, macrophages in the presence of tumor cells and neutrophils, and activated by the immune complexes, showed an induction comprising a significant increase in the amount of secreted IL-1β compared to macrophages incubated with tumor cells and neutrophils only. Thus, there was a statistically significant induction of IL-1β production (a measurable biological response) by FcγRI-expressing macrophages (alone or in combination with tumor cells and neutrophils) activated by immune complexes comprising shed antigen and anti-shed antigen IgG antibody capable of binding FcγRI.

### EXAMPLE 7

Illustrated in this example, are further embodiments of, as well general considerations for, the assay according to the present invention. In another preferred embodiment, a glycomolecule having a composition similar to shed antigen (e.g., per molecule, more than one carbohydrate chain comprising a terminal 2-6 linked sialic acid) may be assayed for its ability, if any, to act as an inducer of the activation of FcγRI-expressing cells; i.e., to induce a measurable biological response. For example, human hepatomas produce gangliosides (e.g., sialyl(α2-6)paragloboside) that may comprise one or more terminal 2-6 linked sialic acids per molecule. The carbohydrate chain may comprise a structure including NeuAcα2-6Galβ1-4GlcNAcβ. The gangliosides having more than one terminal 2-6 linked sialic acids per molecule may be isolated from a hepatoma by homogenizing the tumor tissue, and extracting the homogenate with a mixture (e.g., 30:60:8, v/v/v) of chloroform, methanol, and sodium acetate (0.2M to 0.8M) followed by purification using methods known in the art (e.g., liquid chromatography for separating by size and/or charge). Each purified ganglioside to be tested may then be assayed in the presence of a mAb of an IgG subtype capable of binding FcγRI and having binding specificity for the epitope comprising the terminal 2-6 linked sialic acid; (e.g., a monoclonal antibody having the binding specificity for an epitope comprising NeuAcα2-6Galβ1, such as mAb IB9 or mAb BSRF-S-97). In one illustrative embodiment of this example, the FcγRI-expressing cells are THP-1, an FcγRI-expressing human macrophage cell line. Using similar techniques as previously described herein, THP-1 cells (e.g., seeded at 10,000 cells/well/96 well plate) are contacted with either 100 μl of serum-free tissue culture medium (absence of ganglioside and mAb), or with 100 μl of serum-free tissue culture medium containing the ganglioside (e.g., in a concentration in the range of from about 0.25 ng to about 1.0 ng) and the mAb (e.g., in a concentration in the range of from about 1 pg/ml to about 100 pg/ml) (e.g., presence of the ganglioside and mAb); incubated (e.g., for a time period in the range of from about 2 hours to about 24 hours) in culture at 37° C.; and then the cells from each well are harvested. Cell membranes may then be prepared for tyrosine kinase activity assays, and the assays then performed, in accordance with the literature accompanying the commercial kit for determining tyrosine kinase activity. A measured biological response comprising an induction in tyrosine kinase activity in the presence of the ganglioside and mAb as compared to the level of tyrosine kinase activity measured in the absence of the ganglioside and mAb, is an indication that the ganglioside comprises a shed antigen (and/or an inducer of activation). There are numerous tyrosine kinase inhibitors known to those skilled in the art (e.g., genistein (conc. 0.1 to 100 μM); tyrophostin A23 (conc. 100 to 200 μM); tyrophostin AG126 (conc. 0.015 to 15 μM) staurosporine (conc. 1 to 100 μM); lavendustin A (5 to 50 μM); herbimycin A (5 to 50 μM); erbstatin (0.004 to 4 μM)). In a preferred embodiment, to inhibit a measurable biological response comprising tyrosine kinase activity, or to assay whether a measurable biological response (e.g., induction of cytokine production, or induction of shed antigen production, etc.) is dependent on an induction of tyrosine kinase as a result of activation of the cross-linked cells, the FcγRI-expressing cells may be pre-incubated (e.g., for about 1 hour to about 25 hours, depending on the type of tyrosine kinase inhibitor, and number of cells in the assay) with the tyrosine kinase inhibitor prior to the addition of the immune complexes (or antibody, if the FcγRI-expressing cells secrete shed antigen) in an immunostimulatory amount to activate the FcγRI-expressing cells. Alternatively, the FcγRI-expressing cells may be co-incubated with both the tyrosine kinase inhibitor, and an immunostimulatory amount of immune complexes for activating the FcγRI-expressing cells. As previously described herein, an inhibitor of the measurable biological response will reduce the measurable biological response when the assay method includes the presence of the inhibitor, as compared to the comparative assay method in which the inhibitor is absent (not added).

As apparent to one skilled in the art, and apparent from the descriptions herein, there are various methods for measuring the measurable biological response induced as a result of activation, depending on the activity or molecule which comprises the measurable biological response. For example, an increase in cyclo-oxygenase-2 (COX-2) activity following the activation of the FcγRI-expressing cells, may be assessed by measuring the accumulation of prostaglandin E2 in the cell culture medium by radioimmunoassay or by detecting the COX-2 protein in the cells detected by immunoblot using specific antibodies to COX-2. Cytokines such as TNFα, IL-1α, IL-6, IL-4, and IFN-γ may be measured using commercial ELISA kits, or by measuring the respective cytokine mRNA by semi-quantitative reverse transcription-polymerase chain reaction (e.g., for measuring gene activation) using methods known in the art. MMPs (i.e., MMP-2, MMP-9) may be measured by semi-quantitative reverse transcription-polymerase chain reaction, by quantitative gelatin substrate zymography with densitometry, or by enzyme immunoassay (e.g., ELISA) using methods known in the art. ICAM-1 or other cell adhesion molecules may be measured using commercial immunoassays (e.g., ELISA), by flow cytometry, or by immunohistochemistry using methods known in the art. MCP-1 or RANTES may be measured using commercial immunoassays (e.g., ELISA) or by semi-quantitative reverse transcription-polymerase chain reaction, using methods known in the art. Myeloperoxidase, and elastase, may be measured using commercial immunoassays (e.g., ELISA), or by biochemical characterization (e.g., specific activity), using methods known in the art.

### EXAMPLE 8

In another embodiment, provided are assay kits for performing the assays (methods) according to the present invention. An assay kit for a method of assaying for a measurable biological response inducible by activation of FcγRI-expressing cells (e.g., useful for screening for inhibitors of activation) by immune complexes comprised of shed antigen and anti-shed antigen antibody of an IgG subtype capable of binding FcγRI comprises: a source of shed antigen; and a source of anti-shed antigen antibody of an IgG subtype capable of binding FcγRI. In a more preferred embodiment, the assay kit comprises: a source of shed antigen; a source of anti-shed antigen antibody of an IgG subtype capable of binding FcγRI; and FcγRI-expressing cells. In another embodiment, an assay kit for a method of assaying for a measurable biological response inducible by activation of FcγRI-expressing cells (e.g., useful for screening for inducers of activation) by immune complexes comprised of shed antigen and anti-shed antigen antibody of a IgG subtype capable of binding FcγRI comprises: a source of anti-shed antigen antibody of a IgG subtype capable of binding FcγRI; and FcγRI-expressing cells (the substance being screened as shed antigen or inducer, is provided by the user of the assay kit). This assay kit may further comprise shed antigen which is provided as a control for the assay kit. The shed antigen may be included in the assay method as a positive control for activation; i.e., by contacting it with the anti-shed antigen antibody of an IgG subtype capable of binding FcγRI and the FcγRI-expressing cells in conditions suitable for inducing the measurable biological response for which is being assayed. Thus, if the expected measurable biological response is not detected in the assay, the user should then consider that one or more of the reagents in the assay kit may not be functioning properly; e.g., possibly the source of antibody has been degraded, or the FcγRI-expressing cells may have lost their ability to be activated by the immunostimulatory immune complexes.

In the assay kits in which a source of shed antigen is provided, the shed antigen may comprise shed antigen as a preparation which in a cell-free form (e.g., a lyophilized preparation or a preparation in solution of isolated and purified shed antigen), packaged in a separate container than the container in which is packaged the anti-shed antigen antibody; or may comprise a FcγRI-expressing cell line producing and secreting shed antigen in culture. The latter may comprise tumor cell lines which secrete one or more of sTn-containing mucin, sTn-containing mucin-like glycoprotein, sTn-containing glycolipid, or a combination thereof. For example, there are numerous, commercially available, tumor cell lines known to those skilled in the art which secrete shed tumor mucin. Illustrative examples that have been previously described herein include human tumor lines T-47D and SW620, and murine tumor cell line Met-129. Alternatively, the source of shed antigen and the source of anti-shed tumor antigen antibody may be a single source, e.g., pre-mixed together in one container, in providing pre-formed, immunostimulatory immune complexes which, in the assay method, may be used to contact the FcγRI-expressing cells in activating the cells to induce a measurable biological response. In a preferred embodiment, the shed antigen comprises a submaxillary mucin (e.g., bovine submaxillary mucin) and the anti-shed antigen antibody comprises a murine IgG1 monoclonal antibody having binding specificity for the sTn epitope. In a more preferred embodiment, the murine IgG1 monoclonal antibody has binding specificity for both (or has cross-reactivity with) the sTn epitope and an epitope comprising a terminal alpha 2-6 linked sialic acid other than sTn (as previously des-cribed herein; e.g., substantially comprising NeuAcα2→6Gal). The assay kit may further comprise one or more cell types of FcγRI-expressing cells. More specifically, the FcγRI-expressing cells may comprise one or more of tumor cells, immune effector cells, endothelial cells, or a cell line derived therefrom. Exemplary tumor cell lines (murine or human) have been previously described herein and include both those FcγRI-expressing tumor cells which secrete shed antigen (shed tumor antigen), and those FcγRI-expressing tumor cells which do not secrete detectable amounts of shed antigen. Exemplary endothelial cells have been previously described herein. Such FcγRI-expressing endothelial cells may comprise a cell line (including immortalized cells), or cloned cells that divide for a limited number of passages. For example, FcγRI-expressing endothelial cells may comprise one or more endothelial cell lines comprising EA.hy 926, HUV-EC-C, ECV 304, HMEC-1, HUVEC, and HUVEC-d. Exemplary immune effector cells have been previously described herein. Such FcγRI-expressing immune effector cells may comprise a cell line (including immortalized cells), or cloned cells that divide for a limited number of passages. For example, FcγRI-expressing neutrophils may further comprise one or more of neutrophil cell lines comprising HL-60, NFS-60, and KG-1; FcγRI-expressing macrophages may further comprise one or more of macrophage cell lines (including macrophage-like cell lines) comprising U937, THP-1, and Mono-Mac-6; FcγRI-expressing astrocytes may further comprise one or more of astrocyte cell lines comprising U87, SVG-TH, AsCh-7, UC-11MG, A735, U-251-MG, U-1242 MG, and 132N1; and FcγRI-expressing microglial may further comprise one or more of microglial cell lines comprising CHME 5, and U373 MG. The FcγRI-expressing cells may be stored in a manner of storage selected from the group consisting of: in a solution, lyophilized for reconstitution, frozen, or a combination thereof.

In any of these embodiments, the assay kits may further comprise an isotype control antibody. An isotype control antibody is known to those skilled in the art as an antibody (preferably a monoclonal antibody) which comprises the same subtype as the test antibody, but lacks binding specificity for components in the assay. More specifically, in the present assay kits, an isotype control antibody comprises an antibody of the same subtype as the anti-shed antigen antibody of an IgG subtype capable of binding FcγRI, but lacks binding specificity for shed antigen; thus, the isotype control antibody is incapable of forming immunostimulatory complexes with shed antigen. Therefore, in the presence of the isotype control antibody and the shed antigen, no immune-complex mediated activation of FcγRI-expressing cells occurs. In a preferred embodiment, the isotype control antibody also lacks any binding specificity or cross-reactivity with epitopes on the FcγRI-expressing cells. It is conceivable that if such binding did occur between the isotype control antibody and the FcγRI-expressing cells, there is a chance that a biological response may be induced. Thus, in choosing an isotype control antibody, it should be tested by contacting it with the FcγRI-expressing cells, and assayed for is the presence or absence of induction of a measurable biological response using similar methods as described herein. The preferred isotype control antibody is one which does not induce a measurable biological response when contacted with FcγRI-expressing cells.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

What is claimed is:

1. An assay kit for a method of assaying for a measurable biological response inducible by activation of FcγRI-expressing cells by immune complexes comprising shed antigen and anti-shed antigen antibody of an IgG subtype capable of binding FcγRI, the assay kit comprising:
(a) a source of shed antigen; and
(b) anti-shed antigen antibody of an IgG subtype capable of binding FcγRI.

2. The assay kit according to claim 1, further comprising FcγRI-expressing cells.

3. The assay kit according to claim 1, further comprising an isotype control antibody.

4. The assay kit according to claim 2, further comprising an isotype control antibody.

5. The assay kit according to claim 1, wherein the source of shed antigen comprises a preparation of shed antigen in a cell-free form.

6. The assay kit according to claim 1, wherein the source of shed antigen comprises a cell line comprised of FcγRI-expressing cells which produce and secrete shed antigen in culture.

7. The assay kit according to claim 1, wherein the kit is used for screening a substance for its ability, if any, to inhibit activation of FcγRI-expressing cells.

8. The assay kit according to claim 1, wherein the anti-shed antigen antibody comprises an IgG1 subtype.

9. The assay kit according to claim 5, wherein the shed antigen and anti-shed antigen antibody are pre-mixed together to form immunostimulatory immune complexes.

10. The assay kit according to claim 2, wherein the FcγRI-expressing cells are selected from the group consisting of a tumor cell line, an endothelial cell line, a neutrophil cell line, a macrophage cell line, an astrocyte cell line, a microglial cell line, and a combination thereof.

11. An assay kit for a method of assaying for a measurable biological response inducible by activation of FcγRI-expressing cells by immune complexes comprising shed antigen and anti-shed antigen antibody of an IgG subtype capable of binding FcγRI, the assay kit comprising:
(a) anti-shed antigen antibody of an IgG subtype capable of binding FcγRI; and
(b) FcγRI-expressing cells.

12. The assay kit according to claim 11, further comprising an isotype control antibody.

13. The assay kit according to claim 11, further comprising, as a control, a preparation of shed antigen.

14. The assay kit according to claim 11, wherein the kit is used for screening a substance for its ability, if any, to induce activation of FcγRI-expressing cells.

15. The assay kit according to claim 10, wherein the anti-shed antigen antibody comprises an IgG1 subtype.

16. The assay kit according to claim 10, wherein the FcγRI-expressing cells are selected from the group consisting of a tumor cell line, an endothelial cell line, a neutrophil cell line, a macrophage cell line, an astrocyte cell line, a microglial cell line, and a combination thereof.

17. An in vitro method for screening a substance for ability of the substance, if any, to inhibit activation of FcγRI-expressing cells to induce a measurable biological response, wherein activation is mediated by immune complexes comprising shed antigen and anti-shed antigen antibody of an IgG subtype capable of binding FcγRI, the method comprising:
(a) in a reaction, contacting the substance with immunostimulatory immune complexes and FcγRI-expressing cells, wherein the immunostimulatory immune complexes comprise shed antigen and anti-shed antigen antibody of an IgG subtype capable of binding FcγRI;
(b) measuring the biological response produced from the FcγRI-expressing cells in the presence of the substance;
(c) contacting the immunostimulatory immune complexes and FcγRI-expressing cells together in the absence of the substance in a comparative assay;
(d) measuring a comparative biological response induced from the FcγRI-expressing cells in the absence of the substance in the comparative assay;
(e) comparing the biological response measured in the presence of the substance to the comparative biological response measured from the FcγRI-expressing cells in the absence of the substance in the comparative assay, wherein a decrease in the biological response measured in the presence of the substance as compared to the comparative biological response measured in the absence of the substance indicates that the substance is an inhibitor of activation of FcγRI-expressing cells.

18. The method according to claim 17, wherein the anti-shed antigen antibody comprises an IgG1 subtype.

19. The method according to claim 17, wherein the FcγRI-expressing cells are selected from the group consisting of tumor cells, immune effector cells, endothelial cells, or a combination thereof.

20. The method according to claim 19, wherein the FcγRI-expressing cells are selected from the group consisting of a tumor cell line, an endothelial cell line, a neutrophil cell line, a macrophage cell line, an astrocyte cell line, a microglial cell line, and a combination thereof.

21. The method according to claim 17, wherein step (a) comprises first contacting the substance with the FcγRI-expressing cells in the absence of the immune complexes, and then contacting the FcγRI-expressing cells with the immune complexes.

22. The method according to claim 17, wherein the substance selected to be screened in the assay comprises a substance of unknown pharmacological activity.

23. Use of the substance, found by the method according to claim 17 to be an inhibitor of the activation of FcγRI-expressing cells, as an inhibitor of activation of FcγRI-expressing cells.

24. An in vitro method for screening a substance for ability of the substance, if any, to induce activation of FcγRI-expressing cells to induce a measurable biological response, wherein activation is mediated by immune complexes comprising shed antigen and anti-shed antigen antibody of an IgG subtype capable of binding FcγRI, the method comprising:
(a) in a reaction, contacting the substance with anti-shed antigen antibody of an IgG subtype capable of binding FcγRI, and FcγRI-expressing cells;
(b) measuring the biological response produced from the FcγRI-expressing cells in the presence of the substance;
(c) contacting the anti-shed antigen antibody and FcγRI-expressing cells together in the absence of the substance in a comparative assay;
(d) measuring a comparative biological response induced from the FcγRI-expressing cells in the absence of the substance in the comparative assay;
(e) comparing the biological response measured in the presence of the substance to the comparative biological response measured from the FcγRI-expressing cells in the absence of the substance in the comparative assay, wherein a increase in the biological response measured in the presence of the substance as compared to the comparative biological response measured in the absence of the substance indicates that the substance, in the presence of anti-shed antigen antibody, is an inducer of activation of FcγRI-expressing cells.

25. The method according to claim 24, wherein the anti-shed antigen antibody comprises an IgG1 subtype.

26. The method according to claim 24, wherein the FcγRI-expressing cells are selected from the group consisting of tumor cells, immune effector cells, endothelial cells, or a combination thereof.

27. The method according to claim 26, wherein the FcγRI-expressing cells are selected from the group consisting of a tumor cell line, an endothelial cell line, a neutrophil cell line, a macrophage cell line, an astrocyte cell line, a microglial cell line, and a combination thereof.


SEQUENCE LISTING (2 sequences)

SEQ ID NO: 1 - DNA, 18 bp, Artificial
  synthesized
acaccacaaaggcagtga

SEQ ID NO: 2 - DNA, 18 bp, Artificial
  synthesized
cacccagagaacagtgtt